(19) United States
(12) Patent Application Publication
(10) Pub. No.: US 2007/0219603 A1
(43) Pub. Date: Sep. 20, 2007

(54) Plant energy instrument

(76) Inventors: Willis Kun Hwa Liu, (US); Gensheng Liu, Jiang Men (CN)

Correspondence Address: LAW OFFICES OF CLEMENT CHENG, 17220 NEWHOPE STREET #127, FOUNTAIN VALLEY, CA, 92708, US

(21) Appl. No.: 11/376,450

(22) Filed: Mar. 15, 2006

(51) Int. Cl. A61F 2/00

(52) U.S. Cl. 607/100

(57) ABSTRACT

A plant energy instrument allows a user to relax under plant electromagnetic field. Plant electrodes form a probe unit connected to wires fitting into a plug. A person may receive plant energy inside a hollow housing having a helmet like configuration. The housing houses an energy field transformer and a charge storage device such as a capacitor. The transformer magnifies the energy field and allows the field to pass through the user. The first switch controls the energy store box switch may activate the transformer, and charge storage device. The storage box can store plant energy when the probe unit is attached to the plant. The storage box is preferably embodied with a glass shell, plant extract liquid and reaction coil. Plant extract can be tea tree oil, and aloe. A second switch may be included to control the counter and timer display to monitor instrument use.

### BACKGROUND OF THE INVENTION

A. Field of the Invention

The present invention relates to a health supplement device. More particularly, the present invention relates to a health enhancement platform for radiating a biological energy of plant for sight hinder treatment.

The present invention is based on a gasification field from the plant gasification matter that can be extracted. The air, water, sunlight and gasification matter has been the basic survival conditions for all kinds of animal and plant.

In the exposition of ancient Chinese medical classics by Xuan Yuan Emperor, the philosophic classics “The Book of Changes”, “The Eight Diagrams” and medical classics of many dynasty, yin and yang, the two opposing principles, in natural, can combine, and produce all the essence and spirit vital elements, whatever fine and great, inside and outside.

Combining medicine and “Changes” theory, according to clinical experience, Mr. Liu gen-sheng, the inventor created the technique of recovering plant gasification matter and transforming nutrition matter into the body, then nutrifying the cells, and repairing the nerve, regulating bio-balance, advanced metabolism.

The gasification matter can be transformed by light catalysis reaction, using the general light wave, changing and transforming gasification matter into body's some accupoints, supply the vital energy to regulate bodily functions. Gasification technique and instrument can raise a new scientific project for modern medicine, and molecular biology, so this is the contribution to the mankind.

The plant gasification matter light catalysis transform and the sight hinder treat instrument implementing the present invention will help change the traditional therapy, this technique and instrument will help many sight hindered patients get very well therapy in the rest, and relax people not requiring any medicine, injection, surgical operation and laser, etc, and this is the therapy (treat method) without side-effect, that conforming the modern medicine requirement by WHO (World Health Organization of United Nations) in the future. This invention will replenish the matter vacancy of medicine treatment.

B. Description of the Prior Art

Plant electromagnetic responses have been monitored for laboratory use similar to that used in polygraph analysis. In the book Secret Life of Plants, Peter Tompkins and Christopher Bird discuss changes in plant physiology measured with electrodes. While some doubt that plants are sentient and intelligent, it is undisputed that plants are alive and have measurable electromagnetic responses to their environment.

Lore has it that ether medium matter can transform a body through acupuncture points by saturating bodily tissue at a molecular and Microbiological level. Other scientific studies have investigated the role of electromagnetism in plant and animal growth. In any case, it would be new and interesting to have controlled interactions with plant electromagnetism, which in a relaxing environment may have a variety of psychological and physical benefits.

### SUMMARY OF THE INVENTION

The myopia treatment instrument is being applied to the principle and technique of “plant gasification matter light catalysis transformation” to transfer plant gasification matter into body's accupoints, through which stimulate the vital energy circulating penetrate the molecular rampart then nutrite the cells, repair the nerve, regulate bio-balancing, advance metabolism, so this instrument is the modern medical equipment for multiple functions such as preventing health and nutriology.

The present invention allows a user to relax under the electromagnetic field of a plant. Plant electrodes can form a probe unit that can be connected to wires fitting into a plug. A person may receive the energy of the plant by placing the user's head inside of a hollow housing having a helmet like configuration. The housing houses an energy field transformer and a charge storage device such as a capacitor. Switches may activate the transformer, and the charge storage device in selective configuration. The store box can store plant energy when the probe unit is attached to the plant. The store box can then store energy after the plug has detached the probe and the probe been removed from the unit. The store box can then power the transformer outside of the immediate presence of a plant. The switches are optionally continuously variable and analog.

The charge storage device is formed as an energy storage box that can be a capacitor of the plant energy. The store box has a preferred embodiment comprising a glass shell, a liquid and a reaction coil. The store box has a positive and negative electrical connection. The liquid is preferably plant extract. One kind of plant extract is tea tree oil, aloe or olive oil.

A transformer mounted in series to the energy storage box receives in the energy from the energy storage box when a switch completes the circuit. The transformer magnifies the energy field and allows the field to pass through the user. The transformer is preferably formed as a hollow tube also having a positive and negative polarity. The first switch controls the energy store box and a second switch may be included to control the counter and timer display.

The user typically lies prone resting a head on a pillow. The user can vertically adjust the housing height to a number of levels so that the user's head is as close as possible to the transformer. The vertical adjustment mechanism preferably comprises a number of notches on a track engaging with a pin mounted on the frame of the housing. A pair of slots disposed on the left and right sides of the user bed allow the housing to be placed in any location over the user from head to toe. Therefore, the housing is adjustable both in the horizontal and vertical dimensions. The housing is preferably placed over the head of the user.

A counter keeps track of the number of uses of the invention, and a timer keeps track of the number of seconds, minutes and hours the invention has been in use. The counter is preferably mounted on the lower surface of the housing. The timer is also preferably mounted on the lower surface of the housing. The lower surface of the housing may include a user interface for operating the housing or for moving the housing. The housing is optionally motor-driven for ease of adjustment.

The device has a preferred method of use. The device is designed to help a nearsighted patient relax in the device and improve vision and myopia. The preferred usage is approximately 20 days, about 30 minutes per day. The exact workings of the medical and scientific theory are unknown, but there have been some anecdotal accounts of vision improvement, some people can raise 3 to 4 times on an international sight list.

Embodiments of the invention will now be described by way of example with reference to the accompanying drawings.

### BRIEF DESCRIPTION OF THE DRAWINGS

FIG. 1 a front perspective view of a plant energy instrument according to the present invention.

FIG. 2 is a front view of the plant energy instrument of FIG. 1.

FIG. 3 is a right side view of the plant energy instrument of FIG. 1.

Similar reference numbers denote corresponding features throughout the attached drawings.

### DETAILED DESCRIPTION OF THE PREFERRED EMBODIMENT

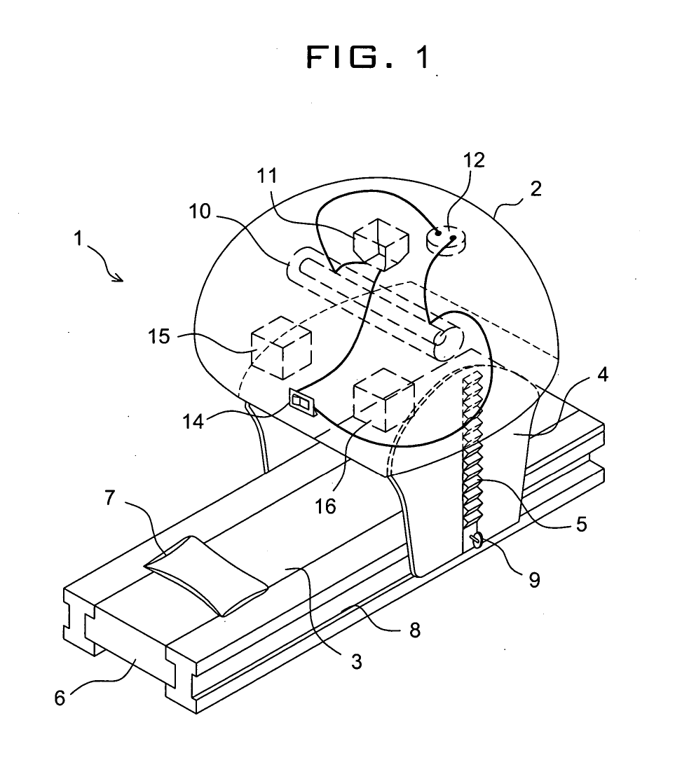

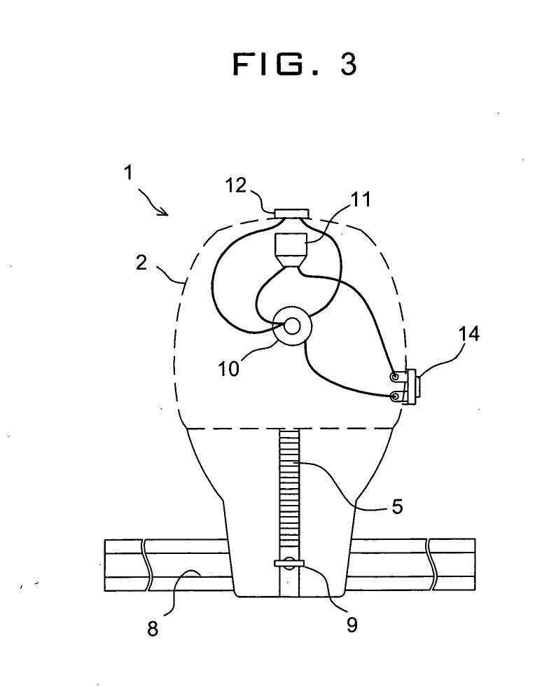

With reference to FIGS. 1 to 3, a plant energy instrument 1 comprises a generally semispherical glass housing or shell 2 suspended over a bed 3 through a pair of support frames 4. The glass shell 2 has an inner frame (not shown) connected to the outer shell 2 for installing components of the instrument leaving side areas open for receiving the frames 4.

Each frame 4 is generally planar and has a number of teeth 5 formed on a vertical outer track adapted to engage a pin mounted on the glass shell 2. The pin pushes against the frame 4 to keep the teeth 5 engaged with the outer glass shell 2 to hold the same and its attachments securely but yield against a forced elevation or depression by a user to adjust the height of the bottom of the glass shell 2 from the bed 3. The shell is preferably placed over the head of the user.

The bed 3 has a cushioned fixed plate 6 where a pillow 7 is laid for relaxation of the user. The bed 3 also includes two opposite side slide rails 8 where the frames 4 are slidably attached at their lower ends. A secure bolt 9 is screwed into a through hole at the lower center of the frame 4 and reaches at an inner wall of the rail 8 so that turning the bolt 9 further will break a sliding movement of the frame 4 while unscrewing the bolt 9 will allow the frame 4 a free sliding in adjusting a horizontal position of the glass shell 2 against the body areas of the user on the bed.

Inside of the glass shell 2 are installed a transformer 10, an energy storage box 11, an input terminal 12 and a switch 14.

The transformer 10 is mounted in series to the energy storage box 11, which supplies the energy when the switch 14 completes a circuit described below. The transformer 10 magnifies the energy field and allows the field to pass through the user. The transformer 10 is preferably formed as a hollow tube also having a positive and negative polarity. The first switch 14 turns the energy storage box 11 on and off and a second switch may be included to control a timer display 15 and a counter 16, which are also included in the glass shell 2 for providing user information.

The transformer 10 relies on the field quantity to absorb the plant gasification matter energy and drive the plant energy instrument 1 with light catalysis. In the transformer 10, general light wave-made “ether medium matter” transforms into a subject's acupuncture points, to penetrate the molecular rampart and supply the additional biological vital energy, to regulate the bodily functions. The transformer 10 is connected at its positive terminal to a positive pole 17 of the energy storage box 11 through a switch 14. The box 11 has a negative pole 18 connected to a negative terminal of the transformer 10. The energy storage box 11 becomes a capacitor of the plant energy or a charge storage device.

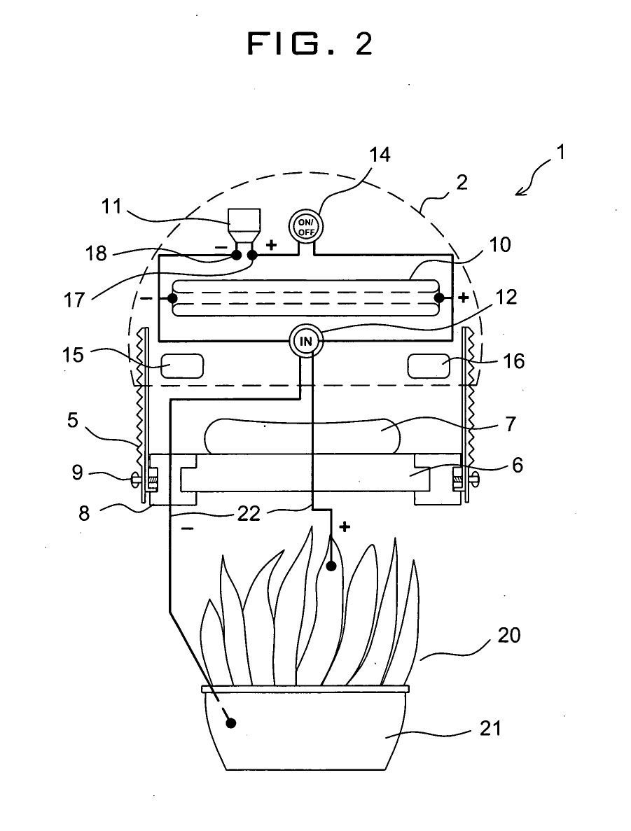

The input terminal 12 is connected across the positive and negative terminals of the transformer 10 to permit connection of the transformer 10 to a plant 20 as shown in FIG. 2 in which a positive wire connect the positive terminal of the transformer 10 to a leaf of the plant 20 while a negative wire at the other terminal penetrates the soil in the pot 21. The wires may be fitted at one ends thereof with a plug for connection with the transformer 10 and plant electrodes at the other ends forming a probe unit 22.

Thus, a complete circuit is formed among the transformer 10, energy storage box 11, and the plant 20. The storage box 11 has a preferred embodiment comprising a glass shell, a liquid and a reaction coil. The storage box 11 has a positive and negative electrical connection. The liquid is preferably plant extract. One kind of plant extract is tea tree oil.

A person may receive the energy of the plant by placing his or her head inside of a recess of the glass shell 2 having a helmet like configuration. The switch 14 may activate the transformer 10, and the charge storage device 11 in selective configuration. The storage box 11 can store plant energy when the probe unit 22 is attached to the plant 20. The storage box 11 can then store energy after the probe unit 22 is unplugged. The storage box 11 can then power the transformer 10 outside of the immediate presence of a plant. The switch 14 is optionally continuously variable and analog.

The user typically lies prone resting a head on a pillow. The user can adjust the shell 2 to a number of vertical levels as well as horizontal positions so that the user's head is as close as possible to the transformer 10.

The counter 16 keeps track of the number of uses of the plant energy instrument 1, and the timer 15 keeps track of the number of seconds, minutes and hours the instrument 1 has been in use. The counter 16 is preferably mounted on the lower surface of the shell 2. The timer 15 is also preferably mounted on the lower surface of the shell 2. The lower surface of the shell 2 may include a user interface for operating the instrument 1 or for moving the shell 2. The glass shell 2 is optionally motor-driven for ease of adjustment.

### Application

The instrument of the present invention has a preferred method of use. The instrument is designed to help a nearsighted patient relax in the device and improve vision and myopia.

Name: The plant gasification matter light catalysis transformation technique, sight hinder treat instrument.

Technique domain: medical appliance health

Background Technique:

The plant gasification matter transformation technique is dependant upon the Chinese medical fuming and steaming with fumigant, by which stimulates the circulation of the blood and causes the muscles and joints to relax, through skin absorption of gas and steam for detumescence and detoxification.

Considering a new treatment concept and molecular biology principles should be used for supplying the modern medicine, the inventor made a plant gasification matter to transform into the body's acupoints, with a circular movement, along the channels, repair cells and nerves like physiotherapy, treat the sight hinder very effectively. This instrument can help patient raise the vision very steadily.

In a treatment program, participants had sight hindrance as myopiahyperopia and astimatism, because they did not care for hygiene health. They work, study, watch TV, operate computer for a long time, and this causes fatigue and the part of people sight hypoplasia as besodity problem.

The sight hinder treatment according to the present invention is the applied technique of biological principle, for general patients, the course of treatment will be twenty times, by the instrument in a relaxed enjoyable environment. The correct position is to put the instrument on the patient's head about 3 centimeters above the “Ying-Tang” acupoint. No touching of the head and face is necessary. After about twenty times of treatment, the patient can remove the sigh fatigue, their sight can be improved evidently. For example, it can raise 3-4 lines on the sight list, particularly some people can change their glasses from deep degrees to lighter degrees, removing their myopia. So, an administrator can supply very simple and safe treatment for many people. To improve their sight hindrance in short term.

### Benefits (Advantages)

1. Material
a. This instrument uses some functional materials to release general light, which doesn't produce any volatile matter, noise, and/or pollution.

2. Production
a. The inventor, himself only, is concerned to have the right interpretation for this instrument, the shape and size can be designed conforming the humanity, and the design model will be in two styles, for enterprise and family use. The plant as mixed materials for plant gasification requirement can be produced in the workshop or laboratory.

3. Supplement
a. This instrument being used is very simple. It does not require any electrical energy and special management. It only needs supplies of some kinds of plant mixed materials. The instrument will be safe, touch free, and producing no noise or pollution. Also, a treatment term is short for improving eyesight.

Therefore, while the presently preferred form of the plant energy instrument has been shown and described, and several modifications thereof discussed, persons skilled in this art will readily appreciate that various additional changes and modifications may be made without departing from the spirit of the invention, as defined and differentiated by the following claims.

### CALL OUT LIST OF ELEMENTS

1: Plant Energy Instrument
2: Housing/Glass Shell
3: Bed
4: Support Frame
5: Teeth
6: Fixed Plate
7: Pillow
8: Slide Rail
9: Secure Bolt
10: Transformer
11: Energy Storage Box
12: Input Terminal
14: Switch
15: Timer
16: Counter
17: Positive Pole
18: Negative Pole
20: Plant
21: Pot
22: Probe Unit

1. A plant energy instrument comprising:
a housing;
a transformer mounted within the housing;
a plug electrically connected to the transformer; and
an electrical probe connected to a plant and connected to the plug, wherein the transformer enhances plant energy.

2. The plant energy instrument of claim 1, wherein the housing is adapted to receive the head of a patient.

3. The plant energy instrument of claim 2, wherein the housing is adapted to receive the head of the patient, and includes a pillow for the patient's head.

4. The plant energy instrument of claim 2 wherein the housing is vertically as well as horizontally adjustable.

5. A plant energy instrument comprising:
a housing;
a transformer mounted within the housing;
a plug electrically connected to the transformer;
an electrical probe connected to a plant and connected to the plug, wherein the transformer enhances plant energy; and
an energy storage box in series with the transformer having a positive and negative polarity, the energy storage box being switch activated.

6. The plant energy instrument of claim 5 wherein the energy storage box has a glass outer shell.

7. The plant energy instrument of claim 5 wherein the energy storage box houses plant extract.

8. The plant energy instrument of claim 5 wherein the energy store box includes a conductive coil.

9. The plant energy instrument of claim 5 wherein the housing has a glass outer shell, wherein the energy storage box houses plant extract, wherein the energy storage box includes a conductive coil.

10. The plant energy instrument of claim 9 wherein the housing is adapted to receive the head of a patient, wherein the housing is vertically adjustable.

11. A plant energy instrument comprising:
a housing adapted to fit over a user;
a transformer mounted within the housing;
a plug electrically connected to the transformer;
an electrical probe connected to a plant and connected to the plug, wherein the transformer enhances and conducts plant electromagnetic energy; and
an energy storage box formed as a capacitor in series with the transformer, wherein the energy storage box is formed as a capacitor.
